(19) United States
(12) Patent Application Publication
(10) Pub. No.: US 2015/0366912 A1
(43) Pub. Date: Dec. 24, 2015

(54) APPLICATION AND PHARMACEUTICAL COMPOSITION OF PREACTIVATED AND DISAGGREGATED SHAPE-CHANGED PLATELETS

(71) Applicants: Yuan-Ji Day, Taoyuan County (TW); Li-Man Hung, New Taipei City (TW); Shiang-Suo Huang, Taipei City (TW); Hsiang-Ruei Liao, Taipei City (TW); Chiou-Mei Lee, Taoyuan County (TW); Jiin-Tarng Liou, Taoyuan County (TW); Fu-Chao Liu, Taoyuan County (TW); Chih-Chieh Mao, Taoyuan County (TW); Polung Yang, Taoyuan County (TW)

(72) Inventors: Yuan-Ji Day, Taoyuan County (TW); Li-Man Hung, New Taipei City (TW); Shiang-Suo Huang, Taipei City (TW); Hsiang-Ruei Liao, Taipei City (TW); Chiou-Mei Lee, Taoyuan County (TW); Jiin-Tarng Liou, Taoyuan County (TW); Fu-Chao Liu, Taoyuan County (TW); Chih-Chieh Mao, Taoyuan County (TW); Polung Yang, Taoyuan County (TW)

(73) Assignee: Chang Gung Medical Foundation, Linkou Branch, Taoyuan county (TW)

(21) Appl. No.: 14/837,926

(22) Filed: Aug. 27, 2015

Related U.S. Application Data
(62) Division of application No. 14/052,755, filed on Oct. 13, 2013.

(51) Int. Cl. A61K 35/19; C12N 11/00

(52) U.S. Cl. CPC A61K 35/19; C12N 11/00

(57) ABSTRACT

Preactivated and disaggregated shape-changed platelets, fixed shape-changed platelets, and a pharmaceutical composition thereof are used for treating acute and emergent inflammatory disease in a dosage of 1×10⁶ to 1×10⁸. Activated platelets release and transfer adhesion factors to the surface of platelet cells, and trap stromal vascularity inflammatory cells from inflammated and damaged place, and the stromal vascularity inflammatory cells are eliminated through the circulatory system to alleviate inflammation. The fixed shape-changed platelets are able to alleviate inflammation and sustainable for longer storage duration.

### CROSS-REFERENCES TO RELATED APPLICATIONS

This is a divisional application of pending U.S. patent application Ser. No. 14/052,755 filed Oct. 13, 2013. The entirety of the above-mentioned patent application is hereby incorporated by reference herein and made a part of specification.

### BACKGROUND OF THE INVENTION

1. Field of the Invention

The present invention relates to shape-changed platelets (SC-PLTs) and a pharmaceutical composition thereof for use in treating acute and emergent inflammatory injuries in a dosage of 1×10⁸ to 1×10⁸.

2. Descriptions of the Related Art

The known platelet-rich plasma (PRP) can be classified into non-activated PRP and activated PRP, and the latter includes PRP hydrogels and emissions or extracts of PRP. PRP hydrogels is fibrinogen released from activated platelets, and it is also used for bone grafts, plastic surgery, dental use and repairing damaged tissues. The emissions or extracts of PRP are rich in growth factors and active proteins, and are considered to promote wound healing and repair damaged bone and joint. The emissions of activated PRP are believed to help repair damaged tissue. However, the related technology, products and patents of the emissions of activated PRP are restricted only in isolation, research and preparation of clottable concentrate of platelet rich in growth factors or related compositions.

Hepatic ischemia-reperfusion (IR) is a topic of great medical and clinical importance, and chronic inflammatory diseases are also injuries of repeated ischemia-reperfusion. The inventors of the subject application indicate that platelets, in addition to a blood clotting function, regulate hepatic inflammation, and prove that shape-changed platelets can reduce acute and emergent inflammatory diseases during hepatic IR.

### SUMMARY OF THE INVENTION

The primary objective of this invention is to provide SC-PLTs and a pharmaceutical composition thereof for use in treating acute and emergent inflammatory diseases in a dosage of 1×10⁶ to 1×10⁸.

Another objective of this invention is to provide fixed-SC-PLTs and a pharmaceutical composition thereof for use in treating acute and emergent inflammatory diseases in a dosage of 1×10⁶ to 1×10⁸.

The fixed-SC-PLTs in a dosage of 1×10⁶ to 1×10⁸ can reduce acute inflammatory injuries, and this proves that the therapeutic effect of the SC-PLTs in reducing acute inflammatory injuries is related to cell surface molecules; and the fixed SC-PLT is sustainable for longer storage duration because it contains no growth factors.

The detailed technology and preferred embodiments implemented for the subject invention are described in the following paragraphs accompanying the appended drawings for people skilled in this field to well appreciate the features of the claimed invention. For the purpose of illustrating the principle of the present invention, the drawings are not necessarily drawn to scale.

### BRIEF DESCRIPTION OF THE DRAWINGS

FIG. 1 is a diagram showing the effect of different dosages of SC-PLTs in treating inflammatory injuries incurred by liver ischemia-reperfusion;

FIG. 2A, show the SC-PLTs used in dermal burn and repair ,which was measured by a ruler;

FIG. 2B, show the SC-PLTs used in dermal burn and repair, which was detected by hematoxylin and eosin (H&E) Staining ;

FIG. 3A shows the SC-PLTs used for increasing viability by reducing inflammatory injuries incurred of sepsis;

FIG. 3B shows the SC-PLTs used in reducing complications of (lung) respiratory failure incurred by sepsis;

FIG. 4A is a schematic diagram showing the SC-PLTs used for increasing viability by reducing damage incurred of ischemic stroke;

FIG. 4B shows the SC-PLTs used for reducing brain infarction incurred by ischemic stroke (n=8, Mean±SE) *, p<0.05;

FIG. 4C shows the SC-PLTs for reducing brain edema incurred by ischemic stroke, the brain water content (%) in the cerebral cortex and striatum (n=8, Mean±SE) *, p<0.05;

FIG. 5 shows the SC-PLTs and fixed-SC-PLTs used for reducing inflammatory injury of liver after 60 minutes ischemia followed by 24 hours reperfusion (n8, Mean±SD) *, p<0.05;

FIG. 6A shows that the SC-PLTs are better than Tyrode's buffer in lowering the abdominal inflammatory cells (CD3e+T lymphocyte) in the CLP sepsis mice;

FIG. 6B shows that the SC-PLTs are better than Tyrode's buffer in lowering abdominal CD11b+monocyte in the CLP sepsis mice; and

FIG. 6C shows that SC-PLTs are better than Tyrode's buffer in lowering abdominal Ly6G+neutrophil in the CLP sepsis mice.

### DETAILED DESCRIPTION OF THE INVENTION

Unless otherwise stated herein, the terms “a (an)”, “the” or the like used in this specification (especially in the Claims hereinafter) shall be understood to encompass both the singular form and the plural form.

The present invention provides shape-changed platelets (SC-PLTs) and a pharmaceutical composition thereof in a dosage of 1×10⁶ to 1×10⁸ for use in treating acute and emergent inflammatory diseases comprising hepatic ischemia-reperfusion inflammatory injuries, sepsis, ischemic stroke, burns, and combinations thereof.

Characteristics of the SC-PLTs in a dosage of 1×10⁶ to 1×10⁸ are as follows. The acute and emergent inflammatory disease is mitigated by activating and transforming the SC-PLT to release cellular adhesion molecules, and the cellular adhesion molecules transfer to the surface of the SC-PLT; the SC-PLTs containing the cellular adhesion molecules are able to adsorb stromal vascularity inflammatory immune cells from inflamed and damaged place, and the inflammatory immune cells are eliminated through the circulatory system.

Preparation of SC-PLTs is as follows: an extremely low dose of an agonist is used to activate the non-activated PRP to obtain disaggregated SC-PLTs.

Fixed-SC-PLTs for treating acute and emergent inflammatory diseases in a dosage of 1×10⁶ to 1×10⁸ are provided, wherein the acute and emergent inflammatory diseases comprise hepatic ischemia-reperfusion inflammatory injuries, sepsis, ischemic stroke, burns, and combinations thereof.

Preparation of fixed-SC-PLTs is as follows: fixing the SC-PLTs with 0.5% paraformaldehyde and washing the released growth factors with a buffer, and suspending the SC-PLTs uniformly in the buffer. The fixed-SC-PLTs in a dosage of 1×10⁶ to 1×10⁸ can reduce acute inflammatory injuries, and this proves that the therapeutic effect of SC-PLTs in reducing acute inflammatory injuries is related to cellular factors on cell surface. Because the fixed SC-PLTs in a dosage of 1×10⁶ to 1×10⁸ contains no cellular growth factors, they are sustainable for longer storage duration and mitigate acute inflammatory injuries.

The present invention uses the fixed-SC-PLTs in a dosage of 1×10⁶ to 1×10⁸ for reducing acute inflammatory injuries. The fixed-SC-PLTs are sustainable for longer storage duration compared to PRP. The application of PRP is limited to cosmetic surgery, arthritis, bone graft materials, and repair medication of wounded tissues; however, the SC-PLTs and fixed-SC-PLTs are of more importance and have broader applications including all inflammatory damages, for example, sepsis, ischemic stroke, burns, hepatic ischemia-reperfusion inflammatory injuries, and surgically induced ischemia-reperfusion. The activated SC-PLTs is useful for reducing acute and emergent inflammatory diseases, and they are also useful for development and design of anti-inflammation drugs.

The present invention provides an anti-inflammation pharmaceutical composition for treating acute and emergent inflammatory diseases, comprising SC-PLTs in a dosage of at least 1×10⁶ to 1×10⁸.

### Examples

[A. Preparation of SC-PLT]

The SC-PLTs are prepared by the following method:

(a) centrifuging mouse whole blood for purification of platelet-rich plasma (PRP) to obtain platelets;
(b) adjusting the concentration of platelets with a buffer to 2.5 to 3×10⁸/mL;
(c) conducting a pre-activation process on the platelets using an agonist including ADP, collagen, thrombin, TRAP, ROS or combinations thereof to obtain the preactivated SC-PLT; and the transforming process of the SC-PLT is as follows: at the pre-activation process the rounded cell membrane is transformed into a pseudopodia form; at this time the preactivated SC-PLT is in a form of proaggregatory suspension particles, and the transmittance of the preactivated SC-PLT decreases to a stable state, and is negative in a blood agglutination test, so as to obtain the SC-PLT; wherein when the platelets react with an agonist in a blood agglutination dosage, the platelets irreversibly become a polymolecular procoagulant from a transition state of small suspension particles , and the transmittance of the platelets in decreases rapidly and temporarily and then increases abruptly to the top (i.e., blood agglutination); wherein the SC-PLTs are preactivated disaggregated platelets, and the SC-PLTs emit biologically active substances stored in cells, a-granuls, dense granules and lysosomes out to cells or transport these substances to the surface of cellular membrane; and the factors transported to the surface of cellular membrane include the cellular adhesion molecule. In one exemplary example the buffer is Tyrode's buffer.

[B. Preparation of Fixed-SC-PLT]

The fixed-SC-PLT is prepared by the following method:

(a) centrifuging mouse whole blood for purification of platelet-rich plasma (PRP) to obtain platelets;
(b) adjusting the concentration of platelets with a buffer to 2.5 to 3×10⁸/mL;
(c) conducting a pre-activation process on the platelets using an agonist including ADP, collagen, thrombin, TRAP, ROS or combinations thereof to obtain the preactivated SC-PLT; and the transforming process of the SC-PLT is as follows: at the pre-activation process the rounded cell membrane is transformed into a pseudopodia form; at this time the preactivated SC-PLT is in a form of proaggregatory suspension particles, and the transmittance of the preactivated SC-PLT decreases to a stable state, and is negative in a blood agglutination test, so as to obtain the SC-PLT; wherein when the platelets react with an agonist in a blood agglutination dosage, the platelets irreversibly become a polymolecular procoagulant from a transition state of small suspension particles, and the transmittance of the platelets decreases rapidly and temporarily and then increases abruptly to the top (i.e., blood agglutination); wherein the SC-PLTs are preactivated disaggregated platelets, and the SC-PLTs emit biologically active substances stored in cells, a-granuls, dense granules and lysosomes out to cells or transport these substances to the surface of cellular membrane; and the factors transported to the surface of cellular membrane include the cellular adhesion molecule;
(d) fixing the SC-PLTs with 0.5% paraformaldehyde and washing the released growth factors with a buffer, and suspending the SC-PLTs uniformly in the buffer. In one exemplary example the buffer is Tyrode's buffer.

[C. Experiments]

(a) Animals: C57BL/6C (B6 albino) male mice were purchased from Charles River Taiwan Branch. The mice, 10 to 12 weeks of age, were fed with food and water ad libitum and were maintained on a 12-h light/dark cycle at 25±1° C. in an animal laboratory.
(b) Route of administration: Tyrode's buffer and SC-PLTs in a dosage of 1×10⁶, 1×10⁷, or 1×10⁸ were injected into mice respectively.
(c) Control group: sham operated mouse control group was used to monitor the surgical procedures, and the damage of sham operated mouse control group was closed to the relative baseline of the non-operated mouse control group.
(d) Statistics: the results were expressed as mean±SE or mean±SD, and P value <0.05 was considered statistically significant.

[D. Results]

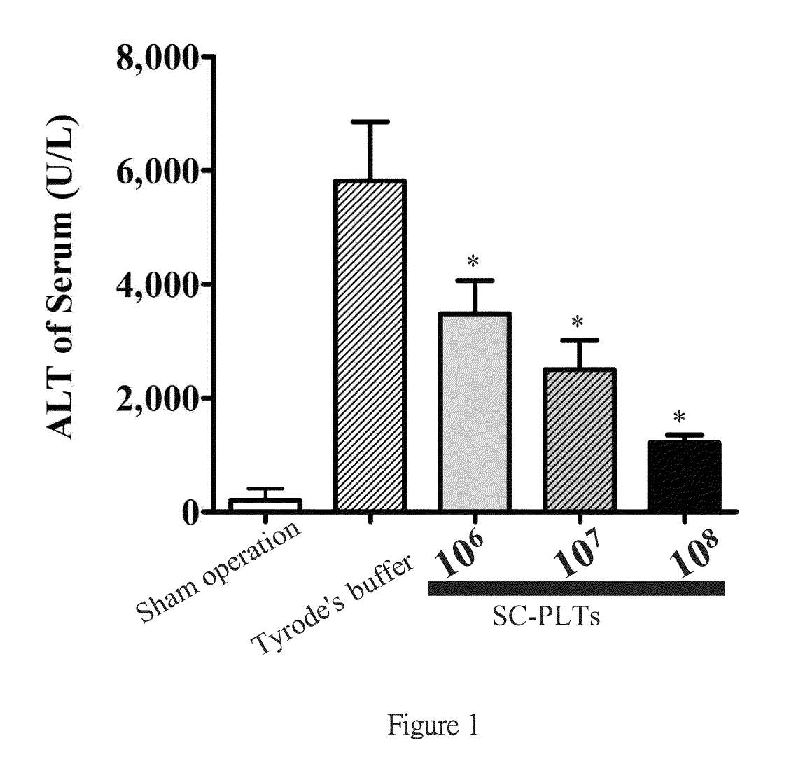

FIG. 1 is a diagram showing the effect of different dosages of SC-PLTs in treating inflammatory injuries incurred by liver ischemia-reperfusion. Tyrode's buffer and the SC-PLTs in a dosage of 1×10⁶, 1×10⁷, and 1×10⁸ respectively were injected into the jugular vein of liver ischemia mice after one hour of ischemia and immediately early reperfusion. The effect of reducing liver inflammatory injuries is positively correlated with the dosage of the SC-PLTs; that is, the SC-PLTs in a dosage of 1×10⁸ had the best therapeutic effect (n≧7, Mean±SD)*, p<0.001).

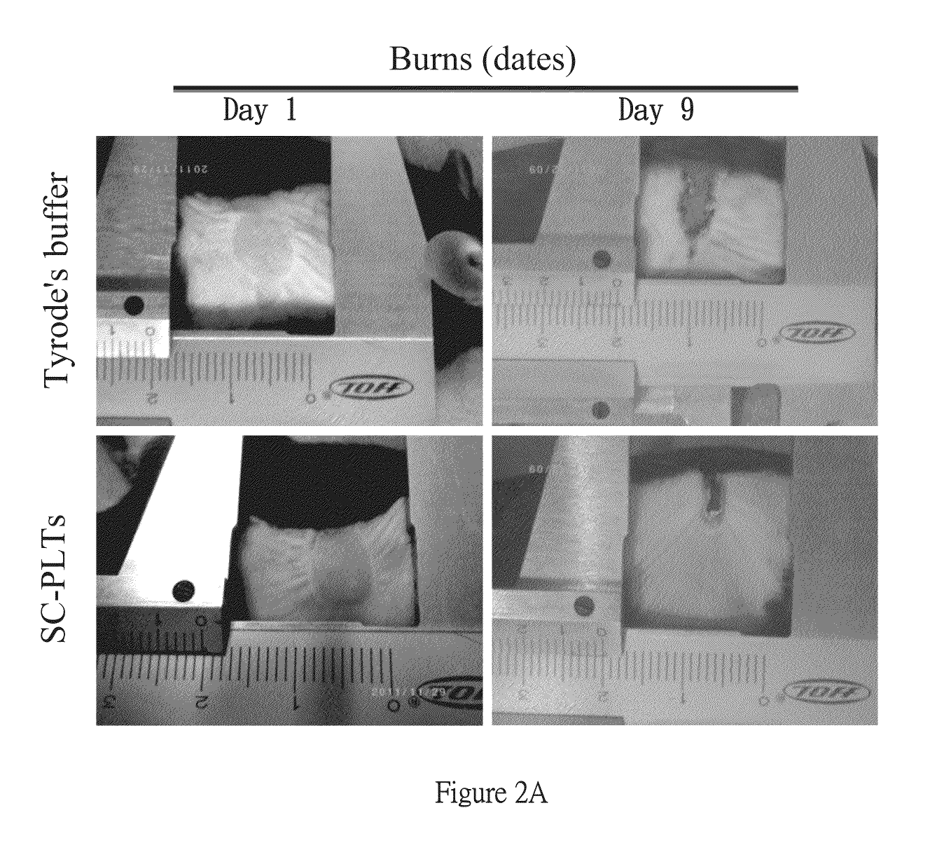

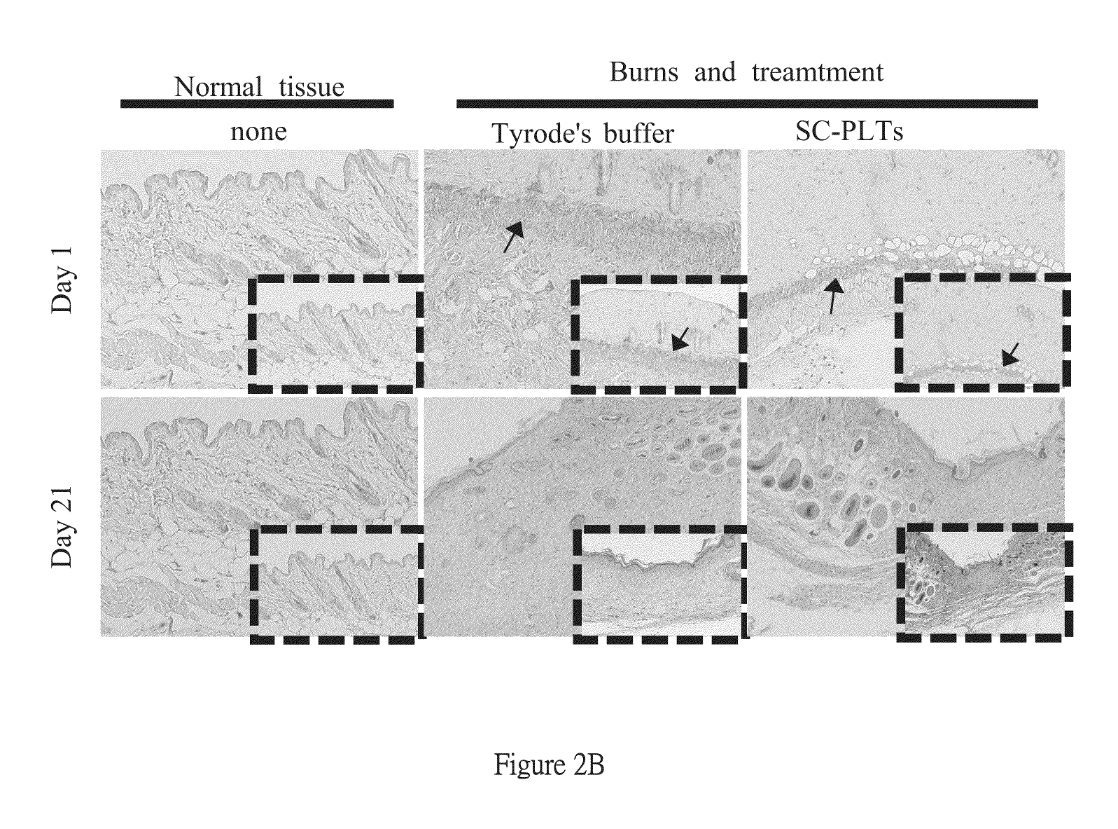

FIG. 2A, 2B show the SC-PLTs used in dermal burn and repair. FIG. 2B shows the H&E stains of burns tissue treated with the SC-PLTs. The arrows indicate the aggregation of inflammatory cells, and the right bottom is the figure on a small scale. An oval shape metal was preheated in boiled water for 5 minutes, and then was used to contact skin for 5 seconds (mice fur is removed using a fur removal cream). The SC-PLTs (or Tyrode's buffer for the control group) were intravenously injected (jugular vein injection in a dosage of 1×10⁸ SC-PLT/200 μL) and in-situ subcutaneously injected (in a dosage of 1×10⁷ SC-PLT/100 μL) into inflammatory area daily and for 5 days continuously. The results proved that the use of SC-PLTs is far better than Tyrode's buffer in healing and repairing of skin burns and reducing acute inflammatory injuries.

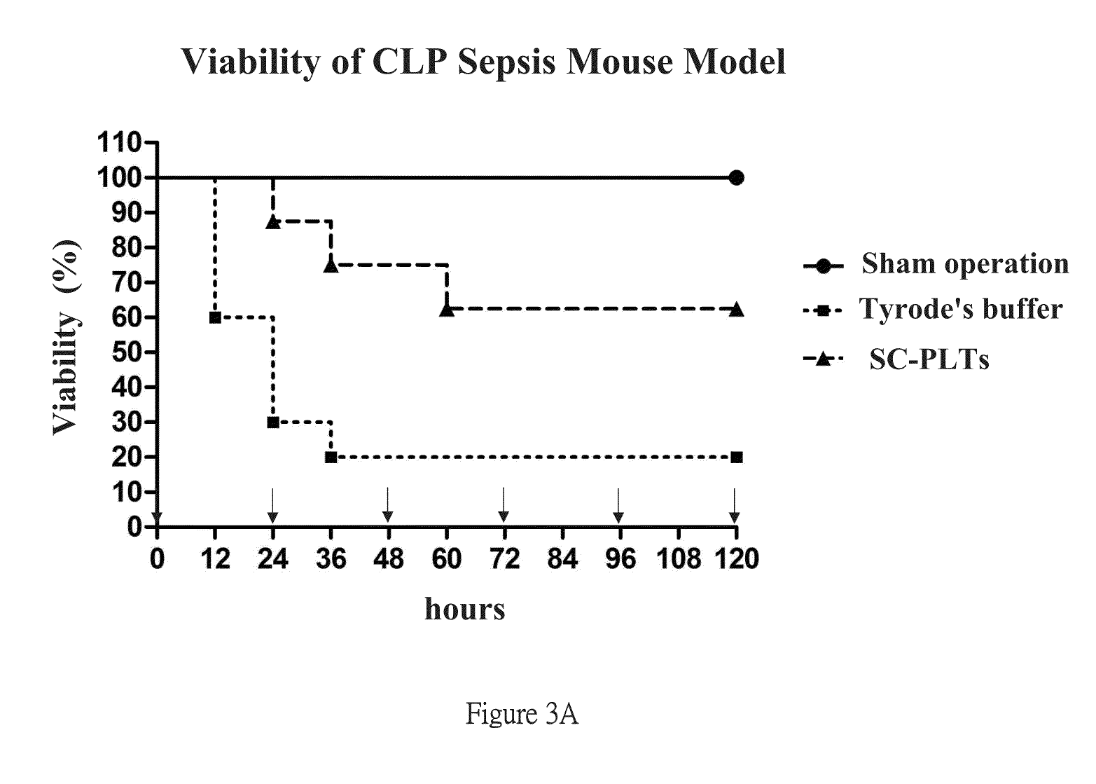

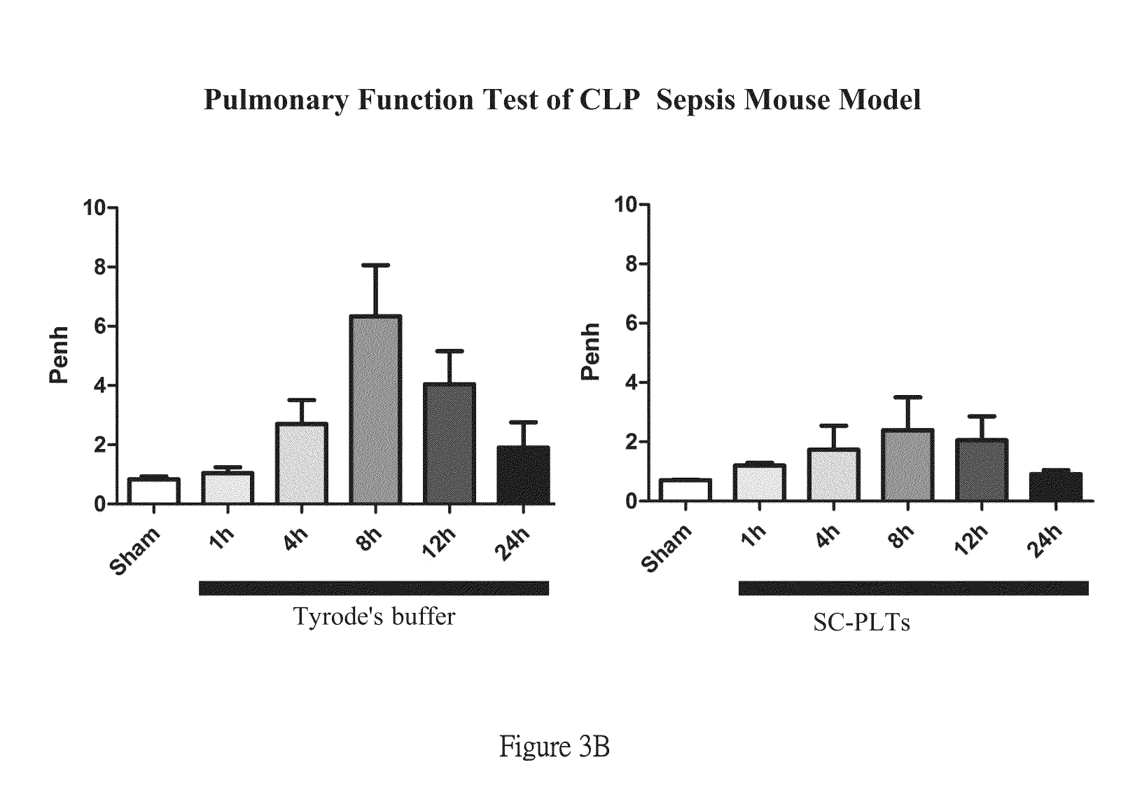

FIG. 3A shows the SC-PLTs used in reducing inflammatory injuries incurred by sepsis. After CLP operation, the mice were intravenously injected and in situ abdominal injected with the SC-PLTs (or Tyrode's buffer for the control group), and the same method and dosage were given every other day. The injection time is indicated as the arrows at 0, 24, 48, 72, 96, 120 hours. Intravenously injection of the SC-PLTs was in a dosage of 1×10⁸ SC-PLT/200 μL and in situ abdominal injected was in a dosage of 1×10⁷ SC-PLT/200 μL (n≧8, Mean±SD)*, p<0.05). The results indicated that the use of the SC-PLTs is far better than Tyrode's buffer in viability in sepsis.

FIG. 3B shows the SC-PLTs used in reducing complications of (lung) respiratory failure incurred by sepsis. Body box was used to measure mice breathing pattern (Penh) and to assess lung failure. The figure shows the curve of time and Pehn after the first dosage treatment given immediately after operation. The results prove that the use of the SC-PLTs reduced post-operation Penh index; that is, SC-PLTs can be used to reduce complications of (lung) respiratory failure incurred by sepsis.

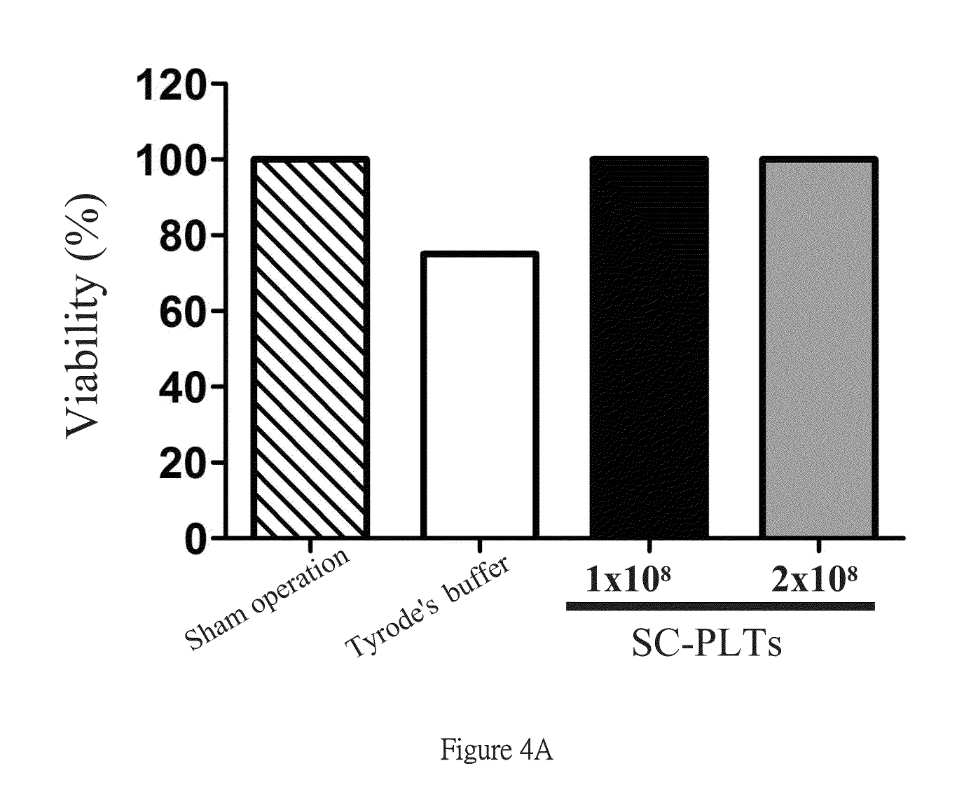

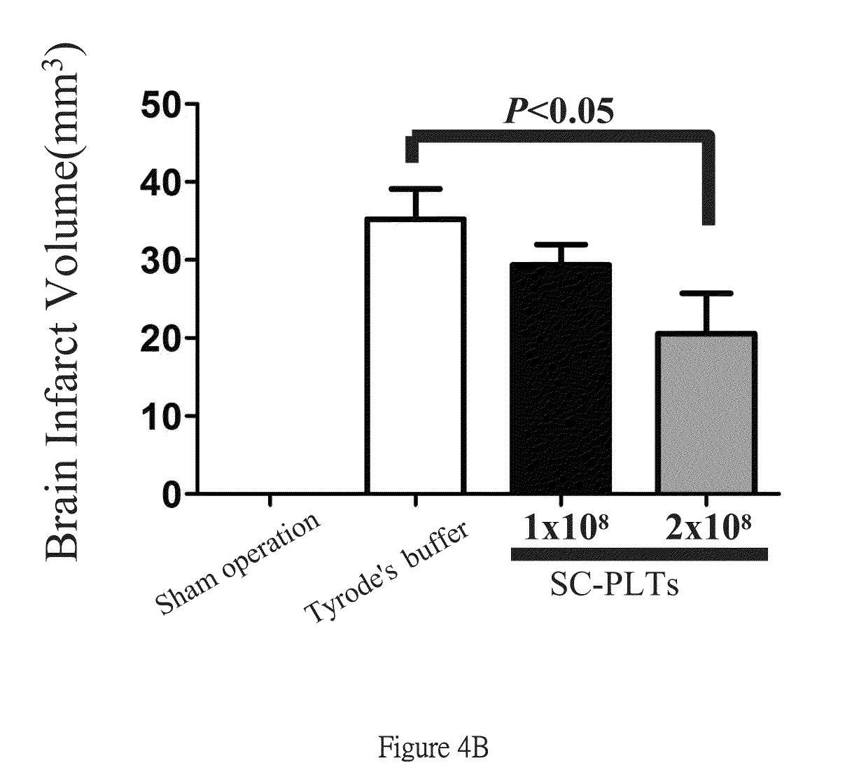

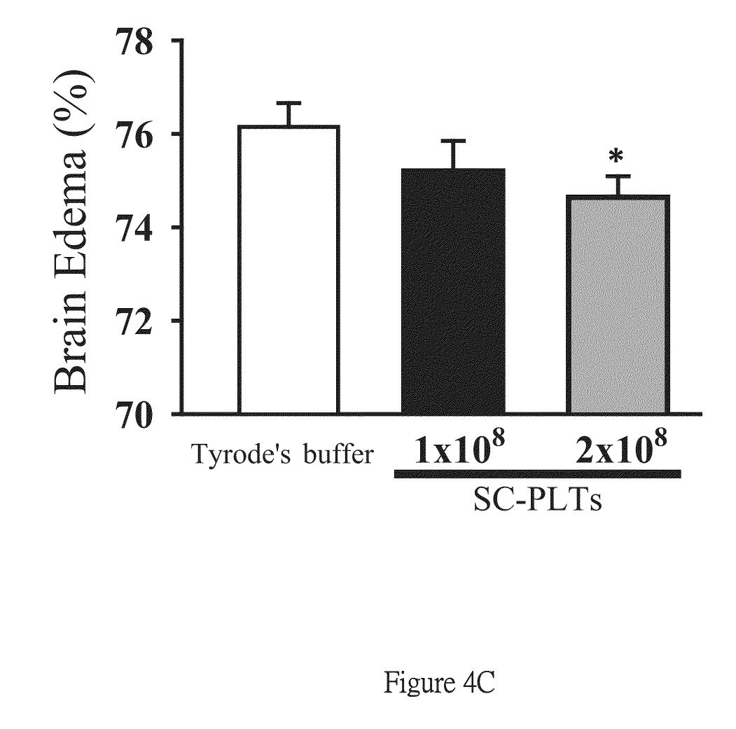

FIG. 4A is a schematic diagram showing the SC-PLTs used for reducing damage incurred by ischemic stroke. The mice were subjected to middle cerebral artery infarction operation, causing ischemia stroke, and then were treated with the SC-PLTs (or Tyrode's buffer for the control group) through jugular vein injection in a dosage of 1×10⁸ and 2×10⁸ SC-PLT/200 μL respectively and treated again with the same method 5 hours after the operation. After 24 hours treatment of single dosage of 1×10⁸ and 2×10⁸ SC-PLT/200 μL, the viability of the mice and brain infarct damage were measured (n=8). The results indicate that the viability is higher in the SC-PLTs group than in the Tyrode's buffer group.

FIG. 4B shows the SC-PLTs used for reducing brain infarction incurred by ischemic stroke (n=8, Mean±SE) *, p<0.05. The results show that the brain infarction damage area is less in the SC-PLTs group than in the control group (Tyrode's buffer). Furthermore, using 2×10⁸ SC-PLTs is better than using 1×10⁸ SC-PLTs and SC-PLT/200 μL in terms of the effect of reducing brain infarction.

FIG. 4C shows the SC-PLTs for reducing brain edema incurred by ischemic stroke, the brain water content (%) in the cerebral cortex and striatum (n=8 , Mean±SE) *, p<0.05. The results show that the brain edema water content (%) incurred by ischemic stroke is lower in the SC-PLTs group than in the control group (Tyrode's buffer). Furthermore, using 2×10⁸ SC-PLTs is better than using 1×10⁸ SC-PLTs and SC-PLT/200 μL in terms of mitigating brain edema.

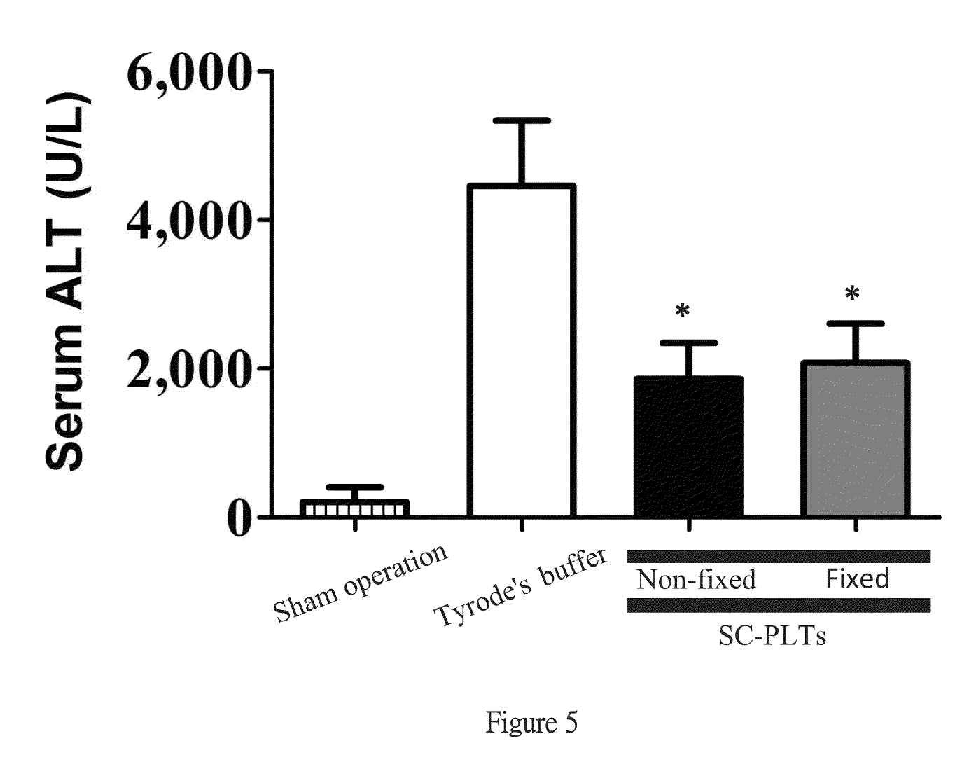

FIG. 5 shows the SC-PLTs and fixed-SC-PLTs used for reducing 60 minutes liver ischemia-reperfusion (n≧8, Mean±SD) *, p<0.05. The result shows that the SC-PLTs and fixed-SC-PLTs are better than Tyrode's buffer in reducing 60 minutes liver ischemia-reperfusion.

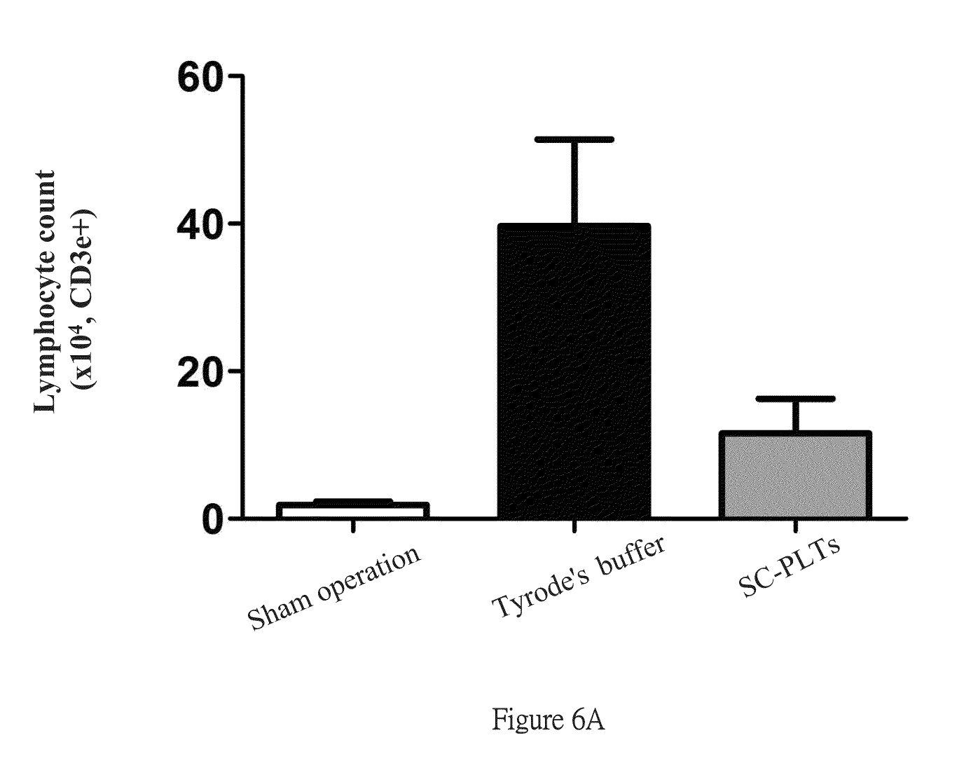

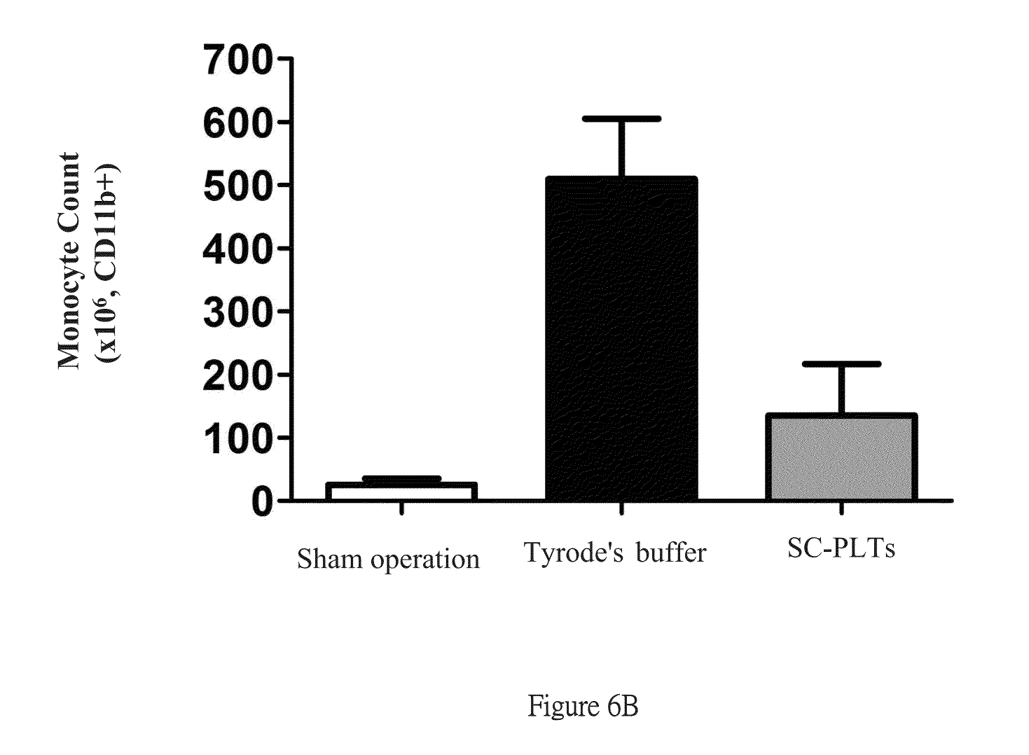

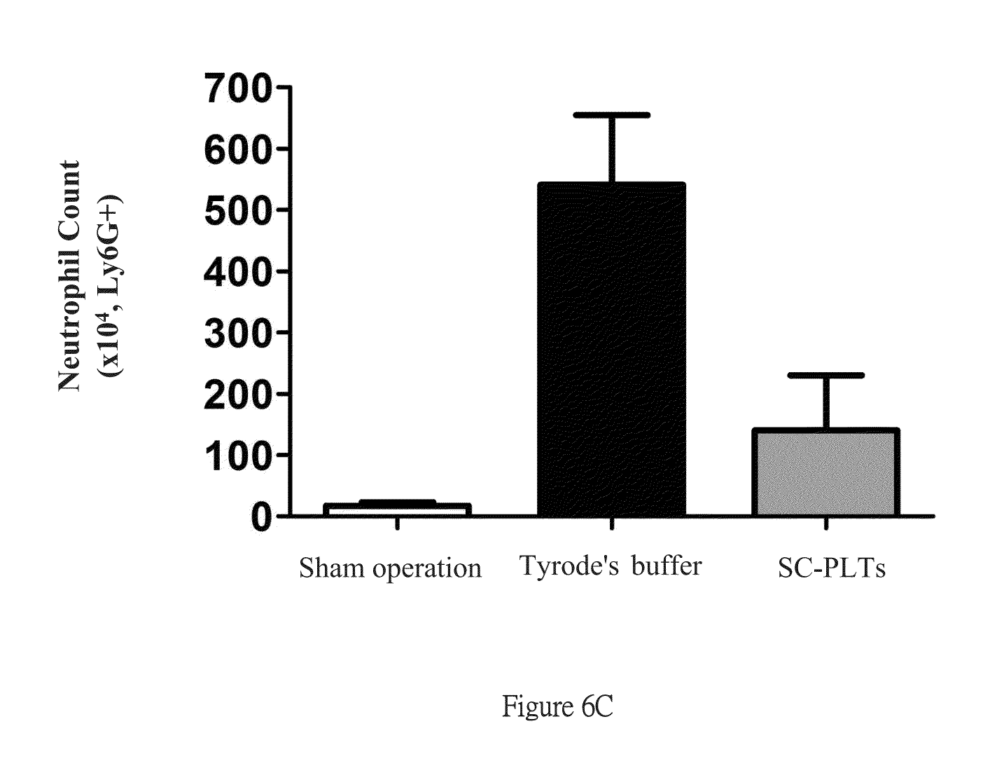

FIG. 6A shows that the SC-PLTs are better than Tyrode's buffer in lowering the abdominal inflammatory cells (CD3e+T lymphocyte) in the CLP sepsis mice.

FIG. 6B shows that the SC-PLTs are better than Tyrode's buffer in lowering abdominal CD 11b+monocyte in the CLP sepsis mice.

FIG. 6C shows that the SC-PLTs are better than Tyrode's buffer in lowering abdominal Ly6G+neutrophil in the CLP sepsis mice.

As showed in FIGS. 1, 4A, 4B, and 4C, comparing liver inflammatory injuries after 24 hours reperfusion, the results prove that the effect of the SC-PLTs is better than that of Tyrode's buffer; furthermore, the effect of 2×10⁸ SC-PLTs/200 μL is better than that of 1×10⁸ SC-PLTs/200 μL in ischemia stroke. Therefore, the effect of reducing acute inflammatory injuries is positively correlated to the injection dosage of the SC-PLTs.

What is claimed is:

1. A fixed SC-PLT in a dosage of 1×10⁶ to 1×10⁸ for treating acute and emergent inflammatory diseases.

2. The fixed SC-PLT as claimed in claim 1, wherein the acute and emergent inflammatory disease comprises sepsis, hepatic ischemia-reperfusion inflammatory injuries, ischemic stroke, burns, and combinations thereof.

3. The fixed SC-PLT as claimed in claim 1, wherein the acute and emergent inflammatory disease is mitigated by activating and transforming the SC-PLT to release cellular adhesion molecules, and the cellular adhesion molecules transfer to the surface of the SC-PLT; the SC-PLT containing the cellular adhesion molecules- is able to adsorb stromal vascularity inflammatory immune cells from inflamed and damaged place, and the inflammatory immune cells (inflammatory leukocyte) are eliminated through the circulatory system; the SC-PLT is able to decrease the concentration of inflammation-related cells including neutrophil, monocyte, and lymphocyte in damaged stromal vascularity; and the fixed SC-PLT is sustainable for longer storage duration because it preserves stable cytoskeleton.

4. The fixed SC-PLT as claimed in claim 1, wherein the SC-PLT is prepared by the following method: (a) centrifuging mouse whole blood for purification of platelet-rich plasma (PRP) to obtain platelets, (b) adjusting the concentration of platelets with a buffer to 2.5 to 3×10⁸/mL, and (c) conducting a pre-activation process on the platelets using an agonist including ADP, collagen, thrombin, TRAP, ROS or combinations thereof to obtain the preactivated SC-PLT; and the transforming process of the SC-PLT is as follows: at the pre-activation process the rounded cell membrane is transformed into a pseudopodia form; at this time the preactivated SC-PLT is in a form of proaggregatory suspension particles, and the transmittance of the preactivated SC-PLT decreases to a stable state, and is negative in a blood agglutination test, so as to obtain SC-PLT; wherein when the platelets react with an agonist in a blood agglutination dosage, the platelets irreversibly become a polymolecular procoagulant from a transition state of small suspension particles, and the transmittance of the platelets decreases rapidly and temporarily and then increases abruptly to the top (i.e., blood agglutination); wherein the SC-PLT is preactivated disaggregated platelets, and the SC-PLT emits biologically active substances stored in cells, a-granuls, dense granules and lysosomes out to cells or transports these substances to the surface of the cellular membrane; and the factors transported to the surface of the cellular membrane include the cellular adhesion molecule; (d) fixing the SC-PLT with 0.5% paraformaldehyde and washing the released growth factors with a buffer, and suspending the SC-PLT uniformly in the buffer.

5. The fixed SC-PLT as claimed in claim 4, wherein the buffer is Tyrode's buffer.
